(19) United States
(12) Patent Application Publication
(10) Pub. No.: US 2023/0330280 A1
(43) Pub. Date: Oct. 19, 2023

(54) PREPARATIONS OF RADIUM-224 AND PROGENIES FOR USE IN RADIONUCLIDE THERAPY IN COMBINATION WITH DNA REPAIR INHIBITORS

(71) Applicant: Oncoinvent AS, Oslo (NO)

(72) Inventors: Jan A. Alfheim, Slattum (NO); Sara Westrøm, Oslo (NO)

(21) Appl. No.: 18/044,665

(22) PCT Filed: Sep. 15, 2021
(86) PCT No.: PCT/EP2021/075301

§ 371 Date: Mar. 9, 2023

(30) Foreign Application Priority Data
Sep. 15, 2020 (EP) 20196215.6

(51) Int. Cl. A61K 51/12; A61K 33/24; A61K 31/5025; A61K 31/502; A61K 31/454; A61K 31/55; A61P 35/00

(52) U.S. Cl. CPC A61K 51/1217; A61K 33/24; A61K 31/5025; A61K 31/502; A61K 31/454; A61K 31/55; A61P 35/00

(57) ABSTRACT

The present invention related to a combination of radium-224 (²²⁴Ra) and/or progeny of ²²⁴Ra, and a DNA repair inhibitor for use in the treatment of cancer. The DNA repair inhibitor can for example be a poly (ADP-ribose) polymerase inhibitor (PARPi), a MGMT inhibitor, a DNA-dependent protein kinase inhibitor (DNA-PK inhibitor), an ataxia telangiectasia and Rad3-related (ATR) kinase inhibitor, an ataxia telangiectasia mutated (ATM) kinase inhibitor, a Wee1 kinase inhibitor, or a checkpoint kinase 1 and 2 (CHK 1/2) inhibitor. The radium-224 (²²⁴Ra) and/or progeny of ²²⁴Ra can be comprised in nano- and/or micro sized particles.

### FIELD

The present invention is related to a combination of radium-224 (²²⁴Ra) and/or progenies of 224Ra, and a DNA repair inhibitor for use in the treatment of cancer. The DNA repair inhibitor can for example be a poly (ADP-ribose) polymerase inhibitor (PARPi), a MGMT inhibitor, a DNA-dependent protein kinase inhibitor (DNA-PK inhibitor), an ataxia telangiectasia and Rad3-related (ATR) kinase inhibitor, an ataxia telangiectasia mutated (ATM) kinase inhibitor, a Wee1 kinase inhibitor, or a checkpoint kinase 1 and 2 (CHK1/2) inhibitor. The radium-224 (²²⁴Ra) and/or progenies of ²²⁴Ra can be comprised in nano- and/or micro sized particles and/or protein or small molecule carriers.

### BACKGROUND

DNA repair inhibitors are known to radiosensitize tumor cells both in vitro and in vivo, and DNA repair inhibitors combined with radionuclide therapy may be promising for treatment of cancers. Chemotherapy and radiation therapy attempt to kill cancer cells by inducing high levels of DNA damage. By inhibiting DNA repair, the efficacy of these therapies can be increased. The DNA inhibitors are mainly known to have radiosensitizing effect with low linear energy transfer (LET) radiation, such as beta-emitting radionuclides. Due to the high LET and complex nature of the damage induced by alpha-emitting radionuclides, such as radium-224 and progenies, it has been considered that there would be less potential of combinations of alpha-emitting radionuclides and DNA repair inhibitors. However, studies have supported a potential of enhanced effect of alpha radionuclide therapy in combination with DNA repair inhibitors. Several of the DNA repair inhibitors approved for clinical use, particularly the PARP inhibitors, could have potentially improved therapeutic outcome within and beyond the disease indications they are currently used for.

One disadvantage that is known for the PARP inhibitors, is a reduced or lack of effect in non-BRCA mutated patients. Combined treatment with a radionuclide therapy might be able to improve the therapeutic potential of current and future DNA repair inhibitors.

The radium isotope ²²⁴Ra (t_{1/2}=3.6 days) has a half-life compatible for use as a therapeutic radionuclide. It decays via multiple alpha- and beta-emitting progeny with shorter half-lives than ²²⁴Ra, with an average of four emitted alpha-particles per complete decay. The complete decay releases a high total energy of 28 MeV, where more than 90% of the energy is associated with the alpha-emissions. Radium-224 and the progenies from the decay of ²²⁴Ra hold promise as therapeutic radionuclides for treatment of cancer.

### SUMMARY

The present invention relates to a combination of a) Radium-224 (²²⁴Ra) and/or progeny of ²²⁴Ra, and b) a DNA repair inhibitor, for use in the treatment of a disease, such as cancer or inflammation.

The DNA repair inhibitor can be selected from the group consisting of a poly (ADP-ribose) polymerase inhibitor (PARPi), a MGMT inhibitor, a DNA-dependent protein kinase inhibitor (DNA-PK inhibitor), an ataxia telangiectasia and Rad3-related (ATR) kinase inhibitor, an ataxia telangiectasia mutated (ATM) kinase inhibitor, a Wee1 kinase inhibitor, and a checkpoint kinase 1 and 2 (CHK1/2) inhibitor.

The progeny of ²²⁴Ra can be selected from the group consisting of ²²⁰Rn, ²¹⁶Po, ²¹²Pb and ²¹²Bi.

The progeny of ²²⁴Ra can be ²²⁰Rn. The progeny of ²²⁴Ra can be ²¹⁶Po. The progeny of ²²⁴Ra can be ²¹²Pb. The progeny of ²²⁴Ra can be ²¹²Bi.

In one or more embodiments of the present invention, the PARPi is selected from the group consisting of Olaparib, Rucaparib, Niraparib, Talazoparib, Veliparib, Pamiparib, CEP 9722, E7016, and 3-Aminobenzamide.

The PARPi can be Olaparib. The PARPi can be Rucaparib. The PARPi can be Niraparib. The PARPi can be Talazoparib. The PARPi can be Veliparib. The PARPi can be Pamiparib. The PARPi can be CEP 9722. The PARPi can be E7016. The PARPi can be 3-Aminobenzamide.

In one or more embodiments of the present invention, the combination for use further comprises nano- and/or micro sized particles.

In one or more embodiments of the present invention, the nano- or microparticles are made of CaCO₃, Ca-Hydroxyaptatite, or fluoroapatite.

In one or more embodiments of the present invention, the CaCO₃ is selected from the group consisting of PEG modified CaCO₃, protein modified CaCO₃, carbohydrate modified CaCO₃, lipid modified CaCO₃, vitamin modified CaCO₃, organic compound modified CaCO₃, polymer modified CaCO₃ and/or inorganic crystal modified CaCO₃.

In one or more embodiments of the present invention, the size of the particle is from 1 nm to 500 μm.

In one or more embodiments of the present invention, the composition is a particle suspension comprising monodisperse or polydisperse particles.

In one or more embodiments of the present invention, the cancer is selected from the group consisting of intraperitoneal cancers, intracranial cancers, pleural cancers, bladder cancers, cardiac cancers, cancers in the subarachnoid cavity, non-cavitary targets such as melanoma, non-small-cell-lung cancer.

In one or more embodiments of the present invention, the treatment is selected from the group consisting of intracavitary therapy or radioembolization.

In one or more embodiments of the present invention, the amount of radionuclide is 1 kBq to 10 GBq per dosing, or with an amount of radionuclide that is 50 MBq to 100 GBq suitable for multidose industrial scale production.

In one or more embodiments of the present invention, the combination or composition comprises one or more selected from the group consisting of a diluent, carrier, surfactant, and/or excipient.

In one or more embodiments of the present invention, a) and b) are administered together or separately.

In one or more embodiments of the present invention, a) and b) are administered within the same day.

In one or more embodiments of the present invention, b) is started one or several days before start of a).

In one or more embodiments of the present invention, b) is initiated one or several days after start of a).

### DETAILED DESCRIPTION

The inventors have surprisingly found that the application of a combination of Radium-224 (²²⁴Ra) and a DNA repair inhibitor is beneficial in the treatment of cancer due to an additive or synergistic effect of the combination. The combination can also have further benefits, such as a less toxicity. The reduced toxicity of the combination can be achieved because less of each of the two elements can be given than what is needed for the single treatment to be efficient. An improved effect of DNA repair inhibitors, such as PARP inhibitors, on non-BRCA mutated cancer patients can also be observed through the combinational use with ²²⁴Ra radiotherapy, as described herein.

Thus, an aspect of the present invention relates to a combination of Radium-224 (²²⁴Ra) and/or one or more progeny or progenies of ²²⁴Ra, and a DNA repair inhibitor, for use as a medicament, such as in the treatment of cancer.

One or more embodiments of the present invention relates to a pharmaceutical composition comprising a) Radium-224 (²²⁴Ra) and/or progeny of ²²⁴Ra, and b) DNA repair inhibitor, such as a poly (ADP-ribose) polymerase inhibitor (PARPi). This composition can be use in the treatment of disease, such as cancer. The two elements, a) Radium-224 (²²⁴Ra) and/or progeny of ²²⁴Ra, and b) DNA repair inhibitor, can also be administered separately, as described herein.

### DNA Repair Inhibitor

The DNA repair inhibitor can be selected from the group consisting of a poly (ADP-ribose) polymerase inhibitor (PARPi), a MGMT inhibitor, a DNA-dependent protein kinase inhibitor (DNA-PK inhibitor), an ataxia telangiectasia and Rad3-related (ATR) kinase inhibitor, an ataxia telangiectasia mutated (ATM) kinase inhibitor, a Wee1 kinase inhibitor, and a checkpoint kinase 1 and 2 (CHK1/2) inhibitor.

One or more embodiments of the present invention relates to a DNA repair inhibitor that is a poly (ADP-ribose) polymerase inhibitor (PARPi). In one or more embodiments of the present invention, the PARPi is selected from the group consisting of Olaparib, Rucaparib, Niraparib, Talazoparib, Veliparib, Pamiparib, CEP 9722, E7016, and 3-Aminobenzamide. The PARPi can be Olaparib. The PARPi can be Rucaparib. The PARPi can be Niraparib. The PARPi can be Talazoparib. The PARPi can be Veliparib. The PARPi can be Pamiparib. The PARPi can be CEP 9722. The PARPi can be E7016. The PARPi can be 3-Aminobenzamide.

One or more embodiments of the present invention relates to a DNA repair inhibitor that is a MGMT inhibitor. In one or more embodiments of the present invention, the MGMT inhibitor is selected from the group consisting of O6 benzylguanine (O6-BG) and O6-(4 bromothenyl) guanine (PaTrin-2 or PAT). The MGMT inhibitor can be O6 benzylguanine (O6-BG). The MGMT inhibitor can be O6-(4 bromothenyl) guanine (PaTrin-2 or PAT).

One or more embodiments of the present invention relates to a DNA repair inhibitor that is a DNA-dependent protein kinase inhibitor (DNA-PK inhibitor). In one or more embodiments of the present invention, the DNA-PK inhibitor is selected from the group consisting of LY294002, NU7441, NU7427, NU7026, NU7163, NU5455, KU-0060648, IC60211 derivatives, CC-115, CC-122, ZSTK474, VX984, VeM3814, and AZD7648. The DNA-PK inhibitor can be LY294002. The DNA-PK inhibitor can be NU7441. The DNA-PK inhibitor can be NU7427. The DNA-PK inhibitor can be NU7026. The DNA-PK inhibitor can be NU7163. The DNA-PK inhibitor can be NU5455. The DNA-PK inhibitor can be KU-0060648. The DNA-PK inhibitor can be IC60211 derivatives. The DNA-PK inhibitor can be CC-115. The DNA-PK inhibitor can be CC-122. The DNA-PK inhibitor can be ZSTK474. The DNA-PK inhibitor can be VX984. The DNA-PK inhibitor can be VeM3814. The DNA-PK inhibitor can be AZD7648.

One or more embodiments of the present invention relates to a DNA repair inhibitor that is an ataxia telangiectasia and Rad3-related (ATR) kinase inhibitor.

One or more embodiments of the present invention relates to a DNA repair inhibitor that is an ataxia telangiectasia mutated (ATM) kinase inhibitor.

One or more embodiments of the present invention relates to a DNA repair inhibitor that is a Wee1 kinase inhibitor.

One or more embodiments of the present invention relates to a DNA repair inhibitor that is a checkpoint kinase 1 and 2 (CHK1/2) inhibitor.

### Radionuclides

The main medical advantages of alpha particle emitting compounds in local therapy in e.g., the intraperitoneal cavity is the shorter range, typically less than 0.1 mm for alphas compared with mm to cm ranges for beta-particles from medical beta emitters. The radionuclide of the present invention can therefore be tailored according to the intended use.

Use of alpha-emitters would in an intracavitary setting reduce risk for toxicity due to irradiation of deeper regions of internal organs like the radiosensitive intestinal crypt cells in the case of intraperitoneal (i.p.) use. Also is the high linear energy transfer of the emitted alpha particles advantageous since very few alpha hits are needed to kill a cell and cellular resistance mechanism like high repair capacity for DNA strand breaks is less of a problem because of the high probability of producing irreparable double strand breaks.

The high effect per decay means less radioactivity is needed reducing the need for shielding of hospital staff and relatives since most alpha- and beta emitters also emits some X-rays and gammas which needs to be shielded against.

In the present context, progeny is understood as the radionuclides that are the result of the decay of a parent radionuclide. Thus, when for example ²²⁴Ra is the parent radionuclide, ²²⁰Rn (the daughter radionuclide), ²¹⁶Po (the granddaughter radionuclide), and ²¹²Pb (the great granddaughter radionuclide). ²²⁰Rn, ²¹⁶Po and ²¹²Pb are therefore all considered progeny radionuclides of ²²⁴Ra.

Thus, in one embodiment is the alpha-emitting radionuclide ²²⁴Ra with the daughter radionuclide ²²⁰Rn, the granddaughter radionuclide ²¹⁶Po, and the great granddaughter radionuclide ²¹²Pb. For the particle of the present invention will these all be comprised by the particle when ²²⁴Ra is the radionuclide.

Thus, the progeny of ²²⁴Ra can be selected from the group consisting of ²²⁰Rn, ²¹⁶Po, ²¹²Pb and ²¹²Bi. The progeny of ²²⁴Ra can be ²²⁰Rn. The progeny of ²²⁴Ra can be ²¹⁶Po. The progeny of ²²⁴Ra can be ²¹²Pb. The progeny of ²²⁴Ra can be ²¹²Bi.

These radionuclides can be combined in the use according to the present invention, so one, two or more of the above-mentioned radionuclides are used in combination. This can happen by natural causes where a radionuclide decays and therefore becomes its natural progeny. Such situation can for example happen when ²²⁴Ra is the parent radionuclide, ²²⁰Rn (the daughter radionuclide), ²¹⁶Po (the granddaughter radionuclide), and ²¹²Pb (the great granddaughter radionuclide). ²²⁰Rn, ²¹⁶Po and ²¹²Pb are therefore all considered progeny radionuclides of ²²⁴Ra and will due to the natural decay of ²²⁴Ra automatically be present in certain amounts.

Two or more radionuclides can be used in combination and be beneficial to have higher amounts than from the natural decay according to the intended use. This can for example happen if ²²⁴Ra and ²¹²Pb are mixed. There will in this situation be a higher level of ²¹²Pb than there would be if purified ²²⁴Ra was used.

The amount of radionuclide used per patient dosage may be in the range of 1 kBq to 10 GBq more preferably 100 kBq to 100 MBq, event more preferably range is 0.5 MBq to 25 MBq. Range dosage can be 10 MBq to 10 GBq per patient dose. Range dosage can be 10 MBq to 5 GBq per patient dose. The ranges can be for beta emitters, alpha emitters or combinations hereof. The ranges can be used for therapy. Dosage will depend on the cancer type, and for example how aggressive the disease is. In one embodiment is the dosage 10-100 kBq/kg, such as 20-50 kBq/kg. In another embodiment is the dosage 10-1000 kBq/kg, such as 25-300 kBq/kg. In a further embodiment the is the dosage 100-500 kBq/kg, such as 150-300 kBq/kg. Dosage range can be 10 MBq to 10 GBq per patient dose. Dosage range can be 10 MBq to 5 GBq per patient dose. The ranges can be for beta emitters, alpha emitters or combinations hereof. The ranges can be for therapy.

In one embodiment of the present invention is the pharmaceutical composition prepared with an amount of radionuclide that is 1 kBq to 10 GBq per dosing. For instance, if 100 patient doses are produced in one batch per day this could be made up of a total of 1-10 GBq divided into 100 single dosing vials or ready to use syringes.

In another embodiment of the present invention is the pharmaceutical composition prepared with an amount of radionuclide that is suitable for multidose industrial scale production e.g., 50 MBq to 100 GBq.

Thus, the compositions of the present invention can be prepared with an amount of radionuclide that is 1 kBq to 10 GBq per dosing or with an amount of radionuclide that is 50 MBq to 100 GBq suitable for multidose industrial scale production.

### Carriers

Radium-224 or any of the radionuclide progenies of radium-224 can be used as a solution with or without a carrier compound for delivery of the radionuclides.

The carrier compound can be a protein-based carrier such as an antibody, antibody fragment, or a peptide. The carrier can also be a vitamin, including folate or folate derivates. The carrier can also be an inorganic particle, including nano-or microparticles of CaCO₃ as described below.

Thus, Radium-224 (²²⁴Ra) and/or the progenies of radium-224 can be prepared as solutions or combined with carrier compounds such as nano-or microparticles, protein or peptides, or small molecules, and be used in combination with DNA repair inhibitors for the indications described herein.

In one or more embodiments of the present invention, the combination for use further comprises nano- and/or micro sized particles, also simply referred to as particles in the present disclosure.

The particles can have a variety of characteristics, and the size of the particles can vary depending on the intended uses and applications. The particles can comprise Radium-224 (²²⁴Ra) and/or the progenies of radium-224 in combination with a degradable compound and optionally additional components such as a phosphorus containing additive or for example a targeting compound such as an antibody.

The degradable compound can vary in sizes from 1 nm to 500 μm. The size can be in the range of 100 nm to 50 μm and further preferably is size in the range of 1-10 μm. In one preferred embodiment is the size 1-10 μm. In another preferred embodiment the size is 100 nm to 5 μm, and in another 10-100 nm.

An aspect relates to one or more particles according to the present invention, which are comprised in a composition for use in combination with a DNA repair inhibitor as treatment of a disease as described herein. The composition may be a particle suspension comprising monodisperse or polydisperse particles comprising a degradable compound and a radionuclide. The particle can also comprise a phosphorus containing additive.

One or more embodiments of the present invention relates to the use of the particles of the present invention, where the radionuclide is either surface labeled by the radionuclide, inclusion labeled as part of particle volume, or a surface labeled particle that after radiolabeling is covered with a layer of material to protect the radiolabeled surfaces and prevent radionuclide release. The particle of the present invention can then become a radionuclide labeled particle whereby a layer of material has been added to cover the original surface to encapsulate the radionuclide.

The surface labelling can be performed as an adsorption of the radionuclide to the crystal particles driven by the affinity of the elements or the labelling can be performed as co-precipitation where additional inorganic compounds aid the precipitation process. A chelator can be use in this process, and the chelator can be incorporated in the particle. The chelator can also be used without a particle, i.e. simply by being used as a carrier itself or as the means for combining a targeting molecule or moiety with the radionuclide.

Thus, the Radium-224 (²²⁴Ra) and/or the progenies of radium-224 in the present invention can be conjugated to a targeting molecule by using bifunctional chelators.

These could be cyclic, linear or branched chelators. Particular reference may be made to the polyaminopolyacid chelators which comprise a linear, cyclic or branched polyazaalkane backbone with acidic (e.g. carboxyalkyi) groups attached at backbone nitrogens.

Examples of suitable chelators include DOTA derivatives such as p-isothiocyanatobenzyl-l,4,7,10-tetraazacyclododecane-1,4,7,10-tetraacetic acid (p-SCN-Bz-DOTA) and the tetra primary amide variant of this DOTA compound, termed TCMC, and DTPA derivatives such as p-isothiocyanatobenzyl-diethylenetriaminepenta-acetic acid (p-SCN-Bz-DTPA), the first being cyclic chelators, the latter linear chelators.

Metallation of the complexing moiety may be performed before or after conjugation of the complexing moiety to the targeting moiety. The targeting moiety can be any of the elements described herein, including antibodies and vitamins.

The radiolabeling procedure will in general be more convenient in terms of time used etc. if the chelator is conjugated to the antibody before the radiolabeling takes place.

An aspect of the present invention relates to a composition comprising a particle comprising a degradable compound and a radionuclide, wherein a phosphorus containing additive is comprised in the composition. The composition can be a suspension of particles. The phosphorus containing additive can be incorporated into the particle. The phosphorus containing additive can be associated with the surface of the particle or be present in the surroundings of the particle, i.e. in the composition or suspension that the particle is part of. Thus, one aspect of the present invention relates to a composition or suspension comprising a particle, wherein the particle comprises a degradable compound, a radionuclide and a phosphorus containing additive, and wherein the phosphorus containing additive is associated with the particle by being present in the composition or suspension. The phosphorus containing additive can be as part of the particle. The presence can be on the surface of the particle. The presence can be as part of the composition or suspension of particles. The presence can also be as part of the particle and as part of the composition or suspension of particles.

One or more embodiments of the present invention relate to a particle suspension which is a mixture of a solid phase and a liquid phase. If used, the phosphorus containing additive may either be in the liquid phase. The containing phosphorus additive can be in the solid phase. The phosphorus containing additive can be in the solid and the liquid phases. In the solid phase the phosphorus containing additive can be on the surface or embedded in the particles or both on the surface or embedded in the solid phase. The solid phase might be made out of nanoparticles, microparticles or a combination those two. The radionuclide may be associated with the surface of the particle or embedded in the volume or bulk of the particle, or both. The solid phase can therefore comprise a particle comprising a degradable compound and a radionuclide, with or without a phosphorus containing additive, but the phosphorus containing additive will always be in the liquid phase if it is not part of the solid phase. The degradable compound, radionuclide and phosphorus containing additive can be any of those disclosed herein.

The phosphorus containing compound may or may not complex radionuclide.

The degradable compound of the present invention can be any compound that can be degraded. The degradation can be done by any route selected from the group consisting of high pH, low pH, temperature, proteases, enzymes, nucleases and/or by cellular processes like endocytosis, which also includes phagocytosis. The degradable compounds can therefore be non-toxic salt or a crystal of a non-toxic salt.

In one or more embodiments of the present invention, the degradable compound can be selected from the group consisting of CaCO₃, MgCO₃, SrCO₃, BaCO₃, calcium phosphates including hydroxyapatite Ca₅(PO₄)₃(OH) and fluoroapatite, and composites with any of these as a major constituent. Major constituent is defined as at least 20% of the total molecular weight of the particle, such as at least 30% of the total molecular weight of the particle, such as at least 40% of the total molecular weight of the particle, such as at least 50% of the total molecular weight of the particle, such as at least 60% of the total molecular weight of the particle, such as at least 70% of the total molecular weight of the particle, such as at least 80% of the total molecular weight of the particle, such as at least 90% of the total molecular weight of the particle, such as at least 95% of the total molecular weight of the particle, such as at least 98% of the total molecular weight of the particle, such as at least 99% of the total molecular weight of the particle.

In one or more embodiments of the present invention, the CaCO₃ is selected from the group consisting of PEG modified CaCO₃, protein modified CaCO₃, carbohydrate modified CaCO₃, lipid modified CaCO₃, vitamin modified CaCO₃, organic compound modified CaCO₃, polymer modified CaCO₃ and/or inorganic crystal modified CaCO₃.

The degradable compound can be MgCO₃ which is selected from the group consisting of PEG modified MgCO₃, protein modified MgCO₃ including mAbs and Fabs, carbohydrate modified MgCO₃, lipid modified MgCO₃, vitamin modified MgCO₃, organic compound modified MgCO₃, polymer modified MgCO₃ and/or inorganic crystal modified MgCO₃.

The degradable compound can be SrCO₃ which is selected from the group consisting of PEG modified SrCO₃, protein modified SrCO₃ including mAbs and Fabs, carbohydrate modified SrCO₃, lipid modified SrCO₃, vitamin modified SrCO₃, organic compound modified SrCO₃, polymer modified SrCO₃and/or inorganic crystal modified SrCO₃.

The degradable compound can be BaCO₃ which is selected from the group consisting of PEG modified BaCO₃, protein modified BaCO₃ including mAbs and Fabs, carbohydrate modified BaCO₃, lipid modified BaCO₃, vitamin modified BaCO₃, organic compound modified BaCO₃, polymer modified BaCO₃ and/or inorganic crystal modified BaCO₃.

The degradable compound can be Ca₅(PO₄)₃(OH) which is selected from the group consisting of PEG modified Ca₅(PO₄)₃(OH), protein modified Ca₅(PO₄)₃(OH) including mAbs and Fabs, carbohydrate modified Ca₅(PO₄)₃(OH), lipid modified Ca₅(PO₄)₃(OH), vitamin modified Ca₅(PO₄)₃(OH), organic compound modified Ca₅(PO₄)₃(OH), polymer modified Ca₅(PO₄)₃(OH) and/or inorganic crystal modified Ca₅(PO₄)₃(OH).

The degradable compound can be fluoroapatite which is selected from the group consisting of PEG modified fluoroapatite, protein modified fluoroapatite including mAbs and Fabs, carbohydrate modified fluoroapatite, lipid modified fluoroapatite, vitamin modified fluoroapatite, organic compound modified fluoroapatite, polymer modified fluoroapatite and/or inorganic crystal modified fluoroapatite.

The composite particles can comprise two or more of these degradable compounds where they combined are a major constituent, as defined above.

The degradable compounds may be used as composites with other salts or proteins or peptides and subject to surface modification by surfactants like oleates and similar.

In a special embodiment, the degradable compounds are used with a compound selected from the group consisting of poly ethylene glycol (PEG) modified particles of the degradable compound or inorganic crystal modified degradable compound.

In a special embodiment the degradable compounds are modified with functional receptor and or antigen binding groups, including monoclonal antibodies and derivatives and vitamins and derivatives allowing receptor or antigen binding of particle to individual target cells and diseased tissues. This means that modifications of the particles relate to the addition of other compounds to degradable compounds. This can be done in various ways, and through interactions such as dipole-dipole interactions, ion-dipole and ion-induced dipole forces, hydrogen bonding, Van der Waals forces, and relative strength of forces.

A chelator can be used, preferentially conjugated to a target affinic molecule, e.g., monoclonal or polyclonal antibody or derivatives of antibody, vitamins or derivatives of vitamins. Used as carriers, these elements will be able to target the Radium-224 (²²⁴Ra) and/or the progenies of radium-224 to the desired target. Thus, Radium-224 (²²⁴Ra) and/or the progenies of radium-224 can be combined with a chelator and/or any of these elements.

Monoclonal antibodies (mAbs), polyclonal antibodies (pAbs), antigen-binding fragments (Fabs) and other types of polypeptides and proteins can be used to include specific targeting. Thus, in the particle, i.e. by adding a specific targeting molecule, the particles will be able to have enhanced affinity for certain target cells in the body. A mAb can for example also be conjugated to a chelator which then has affinity for Radium-224 (²²⁴Ra) and/or the progenies of radium-224. This can be done with or without the use of a particle.

The particles can comprise a phosphorus containing additive. The phosphorus containing additive can be a phosphate, thus becoming a phosphate containing additive. The phosphorus containing additive can also be a phosphonate, thus becoming a phosphonate containing additive.

Phosphonates and phosphonic acids are organophosphorus compounds containing C—PO(OH)₂ or C—PO(OR)₂ groups (where R=alkyl, aryl). Phosphonic acids, typically handled as salts, are generally non-volatile solids that are poorly soluble in organic solvents, but soluble in water and common alcohols. Thus, the various salts and acids of the phosphonates are also considered parts of the definition of phosphonate.

A phosphoric acid, in the general sense, is a phosphorus oxoacid in which each phosphorus atom is in the oxidation state +5, and is bonded to four oxygen atoms, one of them through a double bond, arranged as the corners of a tetrahedron. Removal of the hydrogen atoms as protons H⁺ turns a phosphoric acid into a phosphate anion. Partial removal yields various hydrogen phosphate anions.

The phosphorus containing additive can be a phosphonate. The phosphonate can be a bisphosphonate. The bisphosphonate can be selected from the group consisting of Etidronate, Clodronate, Tiludronate, Pamidronate, Neridronate, Olpadronate, Alendronate, Ibandronate, Risedronate, and Zoledronate. In one or more embodiments of the present invention the bisphosphonate is Etidronate. In one or more embodiments of the present invention the bisphosphonate is Clodronate. In one or more embodiments of the present invention the bisphosphonate is Tiludronate. In one or more embodiments of the present invention the bisphosphonate is Pamidronate. In one or more embodiments of the present invention the bisphosphonate is Neridronate. In one or more embodiments of the present invention the bisphosphonate is Olpadronate. In one or more embodiments of the present invention the bisphosphonate is Alendronate. In one or more embodiments of the present invention the bisphosphonate is Ibandronate. In one or more embodiments of the present invention the bisphosphonate is Risedronate. In one or more embodiments of the present invention the bisphosphonate is Zoledronate.

The phosphonate can be a polyphosphonate. The polyphosphonate can be selected from the group consisting of EDTMP-ethylenediamine tetra(methylene phosphonic acid), DOTMP-1,4,7,10-Tetraazacyclododecane-1,4,7,10-tetrayl-tetrakis(methylphosphonic acid) and DTPMP-diethylenetriaminepenta(methylene-phosphonic acid). In one or more embodiments of the present invention the phosphonate is EDTMP-ethylenediamine tetra(methylene phosphonic acid). In one or more embodiments of the present invention the phosphonate is DOTMP-1,4,7,10-Tetraazacyclododecane-1,4,7,10-tetrayl-tetrakis(methylphosphonic acid). In one or more embodiments of the present invention the phosphonate is DTPMP-diethylenetriaminepenta(methylene-phosphonic acid).

The phosphate containing additives can be selected from the group consisting of orthophosphate, linear oligophosphates and polyphosphates, and cyclic polyphosphates. The polyphosphate can be selected from the group consisting of pyrophosphate, tripolyphosphate and triphosphono phosphate. The phosphorus containing additive can be a cyclic polyphosphate which for example can be sodium hexametaphosphate (SHMP).

The concentrations of phosphonates and or phosphate compounds are 1 microgram to 1000 milligram per ml, such as 0.1 mg to 10 mg per ml of final solution, or 1 microgram to 1000 milligram per gram of particles in the final solution.

The composition of the present invention is preferably an aqueous composition. Thus, in this embodiment the liquid phase is an aqueous phase. The composition can be a saline composition. The composition can also be an alcohol composition. The composition can be a gel-matrix composition. The composition of the present invention can be a suspension of the particles of the present invention.

Thus, the compositions and pharmaceutical compositions of the invention can comprise a diluent, vehicle, carrier solution, surfactant, deflocculant and/or excipient.

Acceptable vehicles and pharmaceutical carriers include but are not limited to non-toxic buffers, fillers, isotonic solutions, solvents and co-solvents, anti-microbial preservatives, anti-oxidants, wetting agents, antifoaming agents and thickening agents etc. More specifically, the pharmaceutical carrier can be but are not limited to normal saline (0.9%), half-normal saline, Ringer's lactate, dissolved sucrose, dextrose, e.g. 3.3% Dextrose/0.3% Saline. The physiologically acceptable vehicle can contain a radiolytic stabilizer, e.g. ascorbic acid, human serum albumin, which protect the integrity of the radiopharmaceutical during storage and shipment.

The particles may be dispersed in various buffers compatible with medical injections, e.g., dissolved salts and/or proteins and/or lipids and or sugars.

The pharmaceutical compositions can comprise a multitude of particles. These can be the same or different.

### Medical Applications

The combinations, such as particles and/or compositions, of the present invention can be used as radiotherapeutic compounds and/or radiotherapeutic mixtures (compositions and solutions).

An aspect of the invention relates to the combinations, such as particles, compositions and/or pharmaceutical compositions of the present invention, for use as a medicament. An aspect of the invention relates to the combinations, such as particles, compositions and/or pharmaceutical compositions of the present invention, for use in the treatment of cancer.

The cancers can be micrometastatic disease including intraperitoneal cancers, intracranial cancers pleural cancers, bladder cancers, cardiac cancers, cancers in the subarachnoid cavity, pericardial cancer. One or more embodiments of the present invention relates to the use according to the invention, wherein the cancer is intraperitoneal cancers. One or more embodiments of the present invention relates to the use according to the invention, wherein the cancer is pericardial cancer.

The cancers can be micrometastatic, non-cavitary presented disease targets such as melanoma, non-small-cell-lung cancer and prostate cancer. One or more embodiments of the present invention relates to the use according to the invention, wherein the cancer is prostate cancer.

Medical uses of the present invention include human or veterinary use in (1) Intracavitary therapy (2) radioembolization (3) radiosynovectomy (4) as a medical device.

Parenteral injection is a term that encompasses at least intravenous (IV), intramuscular (IM), subcutaneous (SC) and intradermal (ID) administration. Thus, one or more embodiments of the present invention relates to the use of particle, composition or pharmaceutical composition of the present invention in an administration that comprises parenteral injection. One or more embodiments of the present invention relates to the use of particle, composition or pharmaceutical composition of the present invention in an administration that comprises intravenous (IV) administration. One or more embodiments of the present invention relates to the use of particle, composition or pharmaceutical composition of the present invention in an administration that comprises intramuscular (IM), administration. One or more embodiments of the present invention relates to the use of particle, composition or pharmaceutical composition of the present invention in an administration that comprises subcutaneous (SC) administration. One or more embodiments of the present invention relates to the use of particle, composition or pharmaceutical composition of the present invention in an administration that comprises intradermal (ID) administration. One or more embodiments of the present invention relates to the use of particle, composition or pharmaceutical composition of the present invention in an administration that comprises intra-tumor administration.

Intracavitary therapy may include treatment of e.g., intraperitoneal cancers, intracranial cancers, pleural cancers, bladder cancers, cardiac cancers, cancers in the subarachnoid cavity. Examples of cavities where the particles may be used is cranial cavity, thoracic cavity, lung cavity, spinal cavity, pelvic cavity, pericardium, pleural cavity, bladder cavity or a combination of these including cancers spreading on the peritoneum or meninges and organs within any of these cavities. In one embodiment of the present invention is the cancer selected from the group consisting of intraperitoneal cancers, intracranial cancers, pleural cancers, bladder cancers, cardiac cancers, and cancers in the subarachnoid cavity. In one embodiment of the present invention is the cancer selected from the group consisting of metastatic cancer, lung cancer, ovarian cancer, colorectal cancer, stomach cancer, pancreatic cancer, breast cancer, neoplastic meningitis, peritoneal cancer, pleural effusion, malignant mesothelioma, breast cancer, sarcomas, brain cancers like glioblastoma and astrocytoma, bladder cancer, and liver cancer. One or more embodiments of the present invention relates to the use according to the invention, wherein the cancer is metastatic cancer. One or more embodiments of the present invention relates to the use according to the invention, wherein the cancer is lung cancer. One or more embodiments of the present invention relates to the use according to the invention, wherein the cancer is ovarian cancer. One or more embodiments of the present invention relates to the use according to the invention, wherein the cancer is colorectal cancer. One or more embodiments of the present invention relates to the use according to the invention, wherein the cancer is stomach cancer. One or more embodiments of the present invention relates to the use according to the invention, wherein the cancer is pancreatic cancer. One or more embodiments of the present invention relates to the use according to the invention, wherein the cancer is breast cancer. One or more embodiments of the present invention relates to the use according to the invention, wherein the cancer is neoplastic meningitis. One or more embodiments of the present invention relates to the use according to the invention, wherein the cancer is peritoneal cancer. One or more embodiments of the present invention relates to the use according to the invention, wherein the cancer is pleural effusion. One or more embodiments of the present invention relates to the use according to the invention, wherein the cancer is pleural effusion. One or more embodiments of the present invention relates to the use according to the invention, wherein the cancer is malignant mesothelioma. One or more embodiments of the present invention relates to the use according to the invention, wherein the cancer is breast cancer. One or more embodiments of the present invention relates to the use according to the invention, wherein the cancer is sarcoma. One or more embodiments of the present invention relates to the use according to the invention, wherein the cancer is brain cancers like glioblastoma and astrocytoma. One or more embodiments of the present invention relates to the use according to the invention, wherein the cancer is bladder cancer. One or more embodiments of the present invention relates to the use according to the invention, wherein the cancer is liver cancer.

An aspect of the invention relates to the combination, such as particles, compositions and/or pharmaceutical compositions of the present invention, for use in the treatment of cancer, wherein the cancer is selected from the group consisting of intraperitoneal cancers, intracranial cancers, pleural cancers, bladder cancers, cardiac cancers, cancers in the subarachnoid cavity, non-cavitary targets such as melanoma, non-small-cell-lung cancer.

In a special embodiment for the use of the combinations of the present is treatment or amelioration of a disease which is an infection or inflammation rather than or in combination with cancer. The inflammation can for example be arthritis.

In one embodiment of the present invention is the infection selected from the group consisting of a bacterial infection and viral infection.

Radioembolization may include treatment of primary or metastatic cancer in an organ e.g., the liver by administering the particles of the present invention to a blood vessel leading to a tumor in the liver or another solid organ infiltrated by tumor tissue.

Radiosynovectomy for joint disorders including chronic inflammations is targeted radiation treatment for painful joint diseases using radioactive substances. Its use includes treatment of hemophilic arthritis.

Today it is based on beta-particle emitting compounds used for inflammatory or rheumatoid diseases, or synovial arthrosis of various joints, in particular of the knee, hand and ankle. The particles described herein which are degradable could be very useful in radiosynovectomy.

The administered is preferably done by local injection, e.g. intracavitary. In a special embodiment the injection is directly into a tumor.

Another aspect of the present invention relates to a method of treatment or amelioration comprising administration of the combinations of the present invention to an individual in need thereof.

The combinations and compositions of the present invention can be suitable parenteral use, for instance intravenous intracavitary and/or intratumor injections. The Radium-224 (²²⁴Ra) and/or progeny of ²²⁴Ra will typically be administered in using and of these administration patterns, and the DNA repair inhibitor will typically be administered orally. Thus, in one or more embodiments of the present invention the DNA repair inhibitor is administered orally while the ²²⁴Ra and/or progeny of ²²⁴Ra is administered in a different route.

In an aspect of the present invention is the particle according to the present invention a medical device or is comprised in a medical device.

A medical device is any instrument, apparatus, appliance, software, material or other article, whether used alone or in combination, including the software intended by its manufacturer to be used specifically for diagnostic and/or therapeutic purposes and necessary for its proper application, intended by the manufacturer to be used for human beings for the purpose of: Diagnosis, prevention, monitoring, treatment or alleviation of disease; Diagnosis, monitoring, treatment, alleviation of or compensation for an injury or handicap; Investigation, replacement or modification of the anatomy or of a physiological process; Control of conception; and which does not achieve its principal intended action in or on the human body by pharmacological, immunological or metabolic means, but which may be assisted in its function by such means

Medical devices vary according to their intended use and indications. Examples range from simple devices such as tongue depressors, medical thermometers, and disposable gloves to advanced devices such as computers which assist in the conduct of medical testing, implants, and prostheses.

According to the FDA is medical device “an instrument, apparatus, implement, machine, contrivance, implant, in vitro reagent, or other similar or related article, including a component part, or accessory which is: recognized in the official National Formulary, or the United States Pharmacopoeia, or any supplement to them, intended for use in the diagnosis of disease or other conditions, or in the cure, mitigation, treatment, or prevention of disease, in man or other animals, or intended to affect the structure or any function of the body of man or other animals, and which does not achieve any of its primary intended purposes through chemical action within or on the body of man or other animals and which is not dependent upon being metabolized for the achievement of any of its primary intended purposes.”

The present particles are not being metabolized nor do they have significant chemical action within the body. The particles are carriers of radioactivity that are designed not be metabolized or have any chemical action within the body, and this allows for radiotherapy with very limited unwanted side-effects, such as toxicity.

Thus, in one embodiment is the term “medical device” understood as FDAs definition above.

In one or more embodiments of the present invention, a) and b) are administered together or separately.

The combinations of a) Radium-224 (²²⁴Ra) and/or progeny of ²²⁴Ra, and b) a DNA repair inhibitor can be administered within the same day.

In one or more embodiments of the present invention, b) is started one or several days before start of a).

In one or more embodiments of the present invention, b) is initiated one or several days after start of a).

### General

It should be understood that any feature and/or aspect discussed above in connections with the compounds according to the invention apply by analogy to the methods described herein.

The following examples are provided below to illustrate the present invention. They are intended to be illustrative and are not to be construed as limiting in any way.

### BRIEF DESCRIPTION OF THE FIGURES

FIG. 1: SKOV-3 cells treated with the combination of one-point concentration of one drug with escalating concentrations of the combining drug resulted in synergistic interaction of ²²⁴Ra and (A) niraparib and (B) olaparib that were time point and dose dependent as illustrated by the CI grayscale and number matrix.

### EXAMPLES

### Example 1. Production of ²²⁴Ra

The ²²⁴Ra-generator was prepared by mixing a ²²⁸Th source with an actinide resin and loading it on a column. A source of ²²⁸Th in 1 M HNO₃ was purchased from Eckert & Ziegler (Braunschweig, Germany) or Oak Ridge National Laboratory (TN, USA), and an actinide resin based on the DIPEX® Extractant was acquired from Eichrom Technologies LLC (Lisle, IL) in the form of a pre-packed cartridge of 2 mL. The material in an actinide resin cartridge was extracted and the resin was preconditioned with 1 M HCl (Sigma-Aldrich). A slurry of approximately 0.25 mL actinide resin, 0.25 mL 1 M HCl and 0.1 mL ²²⁸Th in 1 M HNO₃ was prepared in a vial (4 mL vial, E-C sample, Wheaton, Millville, NJ) and incubated with gentle agitation for immobilization of ²²⁸Th for 4 h at room temperature and let to rest for a few days. The generator column was prepared in a 1 mL filtration column (Isolute SPE, Biotage AB, Uppsala, Sweden) by first applying 0.2 mL of inactive actinide resin, before the portion containing ²²⁸Th was loaded on top. The inactive resin was introduced in the bottom of the column to serve as a catcher layer if ²²⁸Th was released during operation of the generator. Later, the capacity of the generator was increased. A slurry consisting of 0.4 mL actinide resin, 0.5 mL ²²⁸Th in 1 M HNO₃ and 0.5 mL 1 M HCl was prepared as described above, before it was loaded onto the generator column.

Radium-224 could be eluted regularly from the generator column in 1-2 mL of 1 M HCl. For further purification, the crude eluate from the generator column was loaded directly onto a second actinide resin column. The second column was washed with 1 M HCl. This eluate was evaporated to dryness in a closed system. The vial was placed in a heater block and flushed with N₂-gas through a Teflon tube inlet and outlet in the rubber/Teflon septum on the vial. The acid vapor was led into a beaker of saturated NaOH by a stream of N₂-gas. The radioactive residue remaining after evaporation was dissolved in 0.2 mL or more of 0.1 M HCl. A radioisotope calibrator (CRC-25R, Capintec Inc., Ramsey, NJ) was used to measure the total extracted activity in the process.

### Example 2—Combination of Radiotherapy using Radium-224 with DNA Repair Inhibitor

Ovarian cancer cell lines: ES-2 (clear cell carcinoma) and SKOV-3 (adenocarcinoma), were used to investigate the pharmacodynamic interactions resulting from the paired combination of Radium-224 (²²⁴Ra) with a DNA repair inhibitor exemplified by the poly (ADP-ribose) polymerase inhibitors (PARPi); niraparib and olaparib.

Methodology: The cells in supplemented McCoy's 5A-modified growth medium were plated at a volume of 200 μl and cell concentration of 5,000 cells/ml in black 96-well plates treated for cell culture (Thermo Fisher, MA USA). The cells were incubated for 24 hours under controlled culture conditions of 5% CO₂, 37° C. and 95% humidity in a cell incubator for 22-24 hours.

Thereafter, escalating concentrations of ²²⁴Ra (0.2-150 kBq/ml), niraparib (0.05-13.2 μM) and olaparib (0.15-92.2 μM) were added to the cells (in duplicates) for the assessment of single agent cytotoxicity and determination of the IC₅₀ for each agent. The IC₅₀ of the single agent is a guide for the appropriate choice for the concentrations to use for the paired combinations. Additionally, the cells were simultaneously exposed to paired combinations of ²²⁴Ra with either of the PARPi at escalating concentrations (in duplicates). This was done to assess the pharmacodynamic interactions resulting from the combination of the treatment agents. The cells were further incubated with the treatment agents over a period of 5 days.

At variant timepoints following the addition of the treatment agents i.e. days 3, 4 and 5, cell proliferation was assessed by determining the DNA content in each well which is proportional to the total cell number per well.

The growth medium was aspirated, and the cells were incubated with a dye that binds cellular nucleic acids using the CyQuant NF cell proliferation assay kit (Thermo Fisher) following the manufacturers' protocol. The fluorescence was measured using the Fluoroskan Ascent Fluorometer (Thermo Fisher).

The pharmacodynamic interactions of the output were determined by calculating the combination index (CI) where CI<0.90 is synergistic, CI of 0.90-1.1 is additive and CI>1.1 is antagonistic as described in Table 1.

TABLE 1
Description of the combination index range defining
the pharmacodynamic interactions: synergism, additive
and antagonism, as described by Chou and Talalay.

| CI range | Description |
|---|---|
| <0.1 | Very strong synergism |
| 0.1-0.3 | Strong synergism |
| 0.3-0.7 | Synergism |
| 0.7-0.85 | Moderate synergism |
| 0.85-0.9 | Slight synergism |
| 0.9-1.1 | Additive |
| 1.1-1.2 | Slight antagonism |
| 1.2-1.45 | Moderate antagonism |
| 1.45-3.3 | Antagonism |
| 3.3-10 | Strong antagonism |
| >10 | Very strong antagonism |

Results: Data presented in Table 2 shows the IC₅₀ concentrations of ²²⁴Ra, niraparib and olaparib in ES-2 and SKOV-3 cells. These concentrations elaborate on the sensitivity of each cell line to the different treatments and it is clear to observe that SKOV-3 is less sensitive to PARPi than ES-2 cells.

TABLE 2
IC₅₀ of single agent treatment.

| | Single agent IC₅₀ | | | | | | | | |
|---|---|---|---|---|---|---|---|---|---|
| | Day 3 | | | Day 4 | | | Day 5 | | |
| | ²²⁴Ra | Niraparib | Olaparib | ²²⁴Ra | Niraparib | Olaparib | ²²⁴Ra | Niraparib | Olaparib |
| Cell line | (kBq) | (μM) | (μM) | (kBq) | (μM) | (μM) | (kBq) | (μM) | (μM) |
| ES-2 | 0.77 | 0.69 | 3.51 | 0.33 | 0.78 | 1.23 | 0.66 | 0.62 | 2.21 |
| SKOV-3 | 4.37 | 5.16 | 21.38 | 1.84 | 2.83 | 23.60 | 0.44 | 2.2 | 1.83 |

When paired combinations were evaluated, the IC50 of each individual agent in the combination consequently decreased as shown in Table 3

TABLE 3
IC₅₀ of paired combination treatment

| | Paired combination IC₅₀ | | | | | | | | |
|---|---|---|---|---|---|---|---|---|---|
| | Day 3 | | | Day 4 | | | Day 5 | | |
| | ²²⁴Ra | Niraparib | Olaparib | ²²⁴Ra | Niraparib | Olaparib | ²²⁴Ra | Niraparib | Olaparib |
| Cell line | (kBq) | (μM) | (μM) | (kBq) | (μM) | (μM) | (kBq) | (μM) | (μM) |
| ES-2 | 0.38 | 0.42 | — | 0.22 | 0.25 | — | 0.32 | 0.36 | — |
| | 0.38 | — | 1.43 | 0.13 | — | 0.50 | 0.25 | — | 0.93 |
| SKOV-3 | 5.62 | 2.45 | — | 0.98 | 0.43 | — | 0.50 | 0.22 | — |
| | 3.35 | — | 10.21 | 0.88 | — | 2.67 | 0.26 | — | 0.81 |

An evaluation of the combination indices of paired combinations at concentrations similar to and less than the single agent IC₅₀ revealed that the pharmacodynamic interactions of the pairs were mainly synergistic. This is shown in Table 4 for ES-2 cells and Table 5 for SKOV-3 cells.

TABLE 4
The combination index of the paired combinations of
²²⁴Ra with niraparib and olaparib in the ES-2 cell line.

| | | |
|---|---|---|
| [²²⁴Ra] | [Niraparib] μM | [Olaparib] μM |

| | (kBq) | 0.18 | 0.73 | 0.62 | 2.47 |
|---|---|---|---|---|---|
| Day 3 | 0.16 | 0.99 | 0.66 | 0.89 | 0.55 |
| | 0.65 | 0.58 | 0.52 | 0.57 | 0.61 |
| Day 4 | 0.16 | 0.77 | 0.67 | 0.99 | 0.61 |
| | 0.65 | 0.75 | 0.73 | 0.54 | 0.53 |
| Day 5 | 0.16 | 0.99 | 0.61 | 0.73 | 0.60 |
| | 0.65 | 0.46 | 0.48 | 0.49 | 0.47 |

TABLE 5
The combination index of the paired combinations of ²²⁴Ra
with niraparib and olaparib in the SKOV-3 cell line.

| | | |
|---|---|---|
| [²²⁴Ra] | [Niraparib] μM | [Olaparib] μM |

| | (kBq) | 0.41 | 1.65 | 2.88 | 11.5 |
|---|---|---|---|---|---|
| Day 3 | 0.94 | 1.43 | 0.81 | 0.41 | 0.55 |
| | 3.76 | 0.57 | 0.74 | 0.34 | 0.80 |
| Day 4 | 0.94 | 0.40 | 0.50 | 0.40 | 0.40 |
| | 3.76 | 0.44 | 0.67 | 0.40 | 0.51 |
| Day 5 | 0.94 | 0.80 | 0.57 | 0.57 | 0.63 |
| | 3.76 | 0.56 | 0.68 | 0.63 | 0.60 |

### Example 3—Evaluation of One-point Concentration of One Drug with Escalating Concentrations of the Combining Drug in SKOV-3 Cells

The combination effect of ²²⁴Ra with olaparib and niraparib was evaluated in SKOV-3 at one-point concentrations of one drug with escalating concentrations of the combining drug. Skov-3 has in previous examples been shown to be the least sensitive cell line to all drug tested. The assay used and calculations made were done as described in Example 2. The chosen one-point concentrations were a fraction of the IC50 of each drug at 72 hours to ensure that the level of cytotoxicity of each individual drug was below the inhibitory threshold, in order to capture the interaction effect of the combination. The drug IC50 fractions used were 25% for niraparib, 46% for olaparib and 46% for ²²⁴Ra.

The combination of ²²⁴Ra and the PARPi resulted in synergistic interactions, depending on the timepoint of assessment. The combination of ²²⁴Ra with olaparib was synergistic which emphasises on the benefit of drug combination in tumours with low drug sensitivity.

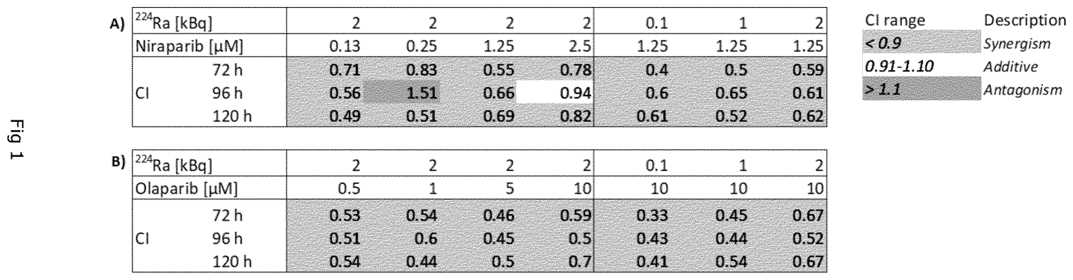

Results are shown in FIG. 1.

### ITEMS

1. A combination of:
a) Radium-224 (224Ra) and/or progeny of ²²⁴Ra, and
b) a DNA repair inhibitor,
for use in the treatment of cancer.

2. The combination for use according to item 1, wherein the a DNA repair inhibitor is selected from the group consisting of a poly (ADP-ribose) polymerase inhibitor (PARPi), a MGMT inhibitor, a DNA-dependent protein kinase inhibitor (DNA-PK inhibitor), an ataxia telangiectasia and Rad3-related (ATR) kinase inhibitor, an ataxia telangiectasia mutated (ATM) kinase inhibitor, a Wee1 kinase inhibitor, and a checkpoint kinase 1 and 2 (CHK1/2) inhibitor.

3. The combination for use according to items 1-2, wherein the progeny of ²²⁴Ra is selected from the group consisting of ²²⁰Rn, ²¹⁶Po, ²¹²Pb and ²¹²Bi.

4. The combination for use according to any of the previous items, wherein the progeny of ²²⁴Ra is ²²⁰Rn.

5. The combination for use according to any of the previous items, wherein the progeny of ²²⁴Ra is ²¹⁶Po.

6. The combination for use according to any of the previous items, wherein the progeny of ²²⁴Ra is ²¹²Pb.

7. The combination for use according to any of the previous items, wherein the progeny of ²²⁴Ra is ²¹²Bi.

8. The combination for use according to any of the previous items, wherein the PARPi is selected from the group consisting of Olaparib, Rucaparib, Niraparib, Talazoparib, Veliparib, Pamiparib, CEP 9722, E7016, and 3-Aminobenzamide.

9. The combination for use according to any of the previous items, wherein the PARPi is Olaparib.

10. The combination for use according to any of the previous items, wherein the PARPi is Rucaparib.

11. The combination for use according to any of the previous items, wherein the PARPi is Niraparib.

12. The combination for use according to any of the previous items, wherein the PARPi is Talazoparib.

13. The combination for use according to any of the previous items, further comprising nano- and/or micro sized particles.

14: The combination for use according to any of the previous items, wherein the carriers are selected from the group consisting of particles, proteins, including antibodies, antibody fragment, or a peptide.

15. The combination for use according to item 13, wherein the nano- or microparticles are made of CaCO₃, or calcium phosphates including Ca-Hydroxyaptatite, or fluoroapatite.

16. The combination for use according to item 13, wherein the nano- or microparticles are made of MgCO₃, SrCO₃ or BaCO₃

17. The combination for use according to item 15, wherein the CaCO₃ is selected from the group consisting of PEG modified CaCO₃, protein modified CaCO₃, carbohydrate modified CaCO₃, lipid modified CaCO₃, vitamin modified CaCO₃, organic compound modified CaCO₃, polymer modified CaCO₃ and/or inorganic crystal modified CaCO₃.

18. The combination for use according to items 14-17, wherein the size of the particle is from 1 nm to 500 μm.

19. The combination for use according to items 14-17, wherein the composition is a particle suspension comprising monodisperse or polydisperse particles.

20. The combination for use according to items 1-19, which is used in the treatment of cancer, and selected from the group consisting of ovarian cancer, colorectal cancer, stomach cancer, liver cancer, peritoneal cancer, pleural cancer, pleural effusion, malignant mesothelioma, pericardial cancer and bladder cancer.

21. The combination for use according to items 1-19, which is used in the treatment of metastatic cancer, and which treatment is selected from the group consisting of sarcomas, osteocarcoma, lung cancer, non-small-cell-lung cancer, pancreatic cancer, breast cancer, neoplastic meningitis, glioblastoma and astrocytoma, melanoma and prostate cancer.

22. The combination for use according to items 1-21, wherein the amount of radionuclide is 1kBq to 10GBq per dosing, or with an amount of radionuclide that is 50 MBq to 100 GBq suitable for multidose industrial scale production.

23. The combination for use according to items 1-22, wherein the combination or composition comprises one or more selected from the group consisting of a diluent, vehicle, carrier solution, surfactant, and/or excipient.

24. The combination for use according to items 1-23, wherein a) and b) are administered together or separately.

25. The combination for use according to items 1-24, wherein a) and b) are administered within the same day.

26. The combination for use according to items 1-24, wherein b) is started one or several days before start of a).

27. The combination for use according to items 1-24, wherein b) is initiated one or several days after start of a).

1. A method of inhibiting a cancer comprising administering a product combination comprising:
a) Radium-224 (224Ra) and/or a progeny of ²²⁴Ra, and
b) a DNA repair inhibitor,

2-19. (canceled)

20. The method according to claim 1, wherein the DNA repair inhibitor is selected from the group consisting of a poly (ADP-ribose) polymerase inhibitor (PARPi), a MGMT inhibitor, a DNA-dependent protein kinase inhibitor (DNA-PK inhibitor), an ataxia telangiectasia and Rad3-related (ATR) kinase inhibitor, an ataxia telangiectasia mutated (ATM) kinase inhibitor, a Wee1 kinase inhibitor, and a checkpoint kinase 1 and 2 (CHK1/2) inhibitor.

21. The method according to claim 1, wherein the progeny of ²²⁴Ra is ²²⁰Rn, ²¹⁶Po, ²¹²Pb or ²¹²Bi.

22. The method according to claim 1, wherein the PARPi is selected from the group consisting of Olaparib, Rucaparib, Niraparib, Talazoparib, Veliparib, Pamiparib, CEP 9722, E7016, and 3-Aminobenzamide.

23. The method according to claim 1, wherein the PARPi is Olaparib or Niraparib.

24. The method according to claim 1, wherein the PARPi is Rucaparib.

25. The method according to claim 1, wherein the PARPi is Talazoparib.

26. The method according to claim 1, further comprising nano- and/or micro sized particles.

27. The method according to claim 26, wherein the nano- and/or microparticles are made of CaCO₃, or a calcium phosphate.

28. The method according to claim 27, wherein the CaCO₃ is selected from the group consisting of PEG modified CaCO₃, protein modified CaCO₃, carbohydrate modified CaCO₃, lipid modified CaCO₃, vitamin modified CaCO₃, organic compound modified CaCO₃, polymer modified CaCO₃ and inorganic crystal modified CaCO₃.

29. The method according to claim 26, wherein the size of the particle is from 1 nm to 500 μm.

30. The method according to claim 26, wherein the product composition is a particle suspension comprising monodisperse or polydisperse particles.

31. The method of claim 1, wherein the cancer is selected from the group consisting of ovarian cancer, colorectal cancer, stomach cancer, liver cancer, peritoneal cancer, pleural cancer, pleural effusion, malignant mesothelioma, pericardial cancer and bladder cancer.

32. The method of claim 1, wherein the cancer is a metastatic cancer, selected from the group consisting of sarcomas, osteocarcoma, lung cancer, non-small-cell-lung cancer, pancreatic cancer, breast cancer, neoplastic meningitis, glioblastoma and astrocytoma, melanoma and prostate cancer.

33. The method of claim 1, wherein the amount of radionuclide is 1 kBq to 10 GBq per dosing, or with an amount of radionuclide that is 50 MBq to 100 GBq suitable for multidose industrial scale production.

34. The method according to claim 1, wherein the product combination further comprises a diluent, vehicle, carrier, carrier solution, surfactant, or excipient.

35. The method according to claim 34, wherein the carrier is selected from particles, proteins, antibodies, antibody fragments, or peptides.

36. The method according to claim 1, wherein a) and b) are administered together or separately.

37. The method according to claim 1, wherein
i) a) and b) are administered within the same day,
ii) b) is started one or several days before start of a), or
iii) b) is initiated one or several days after start of a).
